(19) United States
(12) Patent Application Publication
(10) Pub. No.: US 2023/0050215 A1
(43) Pub. Date: Feb. 16, 2023

(54) CANNABINOID COMPOSITIONS AND PROCESSES FOR MAKING SAME

(71) Applicant: Canateq Holdings Corp., Newport Beach, CA (US)

(72) Inventor: Josh Downey, Rancho Santa Margarita, CA (US)

(21) Appl. No.: 17/661,172

(22) Filed: Apr. 28, 2022

Related U.S. Application Data
(63) Continuation of application No. 16/932,699, filed on Jul. 17, 2020, now abandoned.
(60) Provisional application No. 62/875,113, filed on Jul. 17, 2019.

(51) Int. Cl. A61K 9/51; A61K 31/352; A61K 31/05

(52) U.S. Cl. CPC A61K 9/5192; A61K 31/352; A61K 31/05

(57) ABSTRACT

The present disclosure generally relates to various cannabinoid compositions, including cannabinoid nanoparticle dispersions, processes for preparing these compositions, and methods of using these compositions.

STATEMENT OF RELATED CASES

The present application is a continuation of U.S. application Ser. No. 16/932,699, filed Jul. 17, 2020, which claims the benefit of U.S. Provisional Application Ser. No. 62/875,113, filed Jul. 17, 2019, the entire contents of which are incorporated herein by reference.

### FIELD OF THE INVENTION

The present disclosure generally relates to various cannabinoid compositions, including cannabinoid nanoparticle dispersions, processes for preparing these compositions, and methods of using these compositions.

### BACKGROUND

Cannabinoids are lipophilic and generally have poor solubility in water. Because of their hydrophobic nature, cannabinoids are poorly absorbed systemically from oral dosage forms because of the poor dissolution of cannabinoids in the aqueous environment of gastrointestinal tract. As such, various oral formulations of cannabinoids can exhibit low bioavailability.

Further, incorporating cannabinoids in aqueous based formulations is challenging because of their poor solubility in water. Aqueous-based formulations of cannabinoids have exhibited, among other things, reduced formulation stability, discoloration, unpleasant odor, and unacceptable taste profile.

Oil-based compositions can provide for greater formulation stability. However, when consumed, these compositions can introduce unwanted oils that are not, in some cases, beneficial to the health of the consumer, particularly liver health. As such, oil-based compositions are not desirable in many cases.

Thus, there remains a need for cannabinoid compositions and associated manufacturing processes in which the compositions are compatible in aqueous formulations, exhibit improve bioavailability, and/or contain reduced amounts of undesirable oils.

### BRIEF SUMMARY

Aspects of the present invention relate to various cannabinoid compositions including cannabinoid nanoparticle dispersions. In various embodiments, the cannabinoid nanoparticle dispersions comprise:

a continuous phase comprising a carrier liquid component, and

nanoparticles at least partially dispersed in the carrier liquid component,

wherein the nanoparticles comprise a core material and a coating at least partially encapsulating the core material,

wherein the core material comprises a cannabinoid component comprising a cannabinoid and/or cannabinoid analog and the coating comprises an amphiphilic component, and

wherein the nanoparticles are characterized as having a mean particle size of about 60 nm or less, about 50 nm or less, about 40 nm or less, about 30 nm or less, about 25 nm or less, about 20 nm or less, or about 15 nm or less.

Further aspects relate to other cannabinoid-containing compositions such a drink or beverage composition comprising a dilution (e.g., aqueous dilution) of the nanoparticle dispersion as described herein. Still further cannabinoid-containing compositions of the present invention include various food items, pharmaceuticals, topical compositions, and nutraceuticals comprising the nanoparticle dispersion as described herein or dilution or concentrate thereof.

Various aspects of the present invention are directed to processes for preparing cannabinoid-containing compositions. The processes include those for preparing cannabinoid nanoparticle dispersion including those described herein. In some embodiments, the processes comprise:

exposing a mixture comprising a carrier liquid component, an amphiphilic component, and a cannabinoid component comprising a cannabinoid and/or cannabinoid analog to electromagnetic radiation in a heating zone to form the cannabinoid nanoparticle dispersion comprising nanoparticles at least partially dispersed in a continuous phase comprising at least a portion of the carrier liquid component,

wherein the nanoparticles comprise a core material and a coating at least partially encapsulating the core material, wherein the core material comprises at least a portion of the cannabinoid component and the coating comprises at least a portion of the amphiphilic component, and wherein the nanoparticles are characterized as having a mean particle size of about 60 nm or less, about 50 nm or less, about 40 nm or less, about 30 nm or less, about 25 nm or less, about 20 nm or less, or about 15 nm or less.

Aspects of the present invention also include processes for preparing other cannabinoid-containing compositions such as a drink or beverage composition comprising a dilution (e.g., aqueous dilution) of the nanoparticle dispersion as described herein. In some embodiments, these processes comprise preparing the cannabinoid nanoparticle dispersion as described herein; and diluting the cannabinoid nanoparticle dispersion with a liquid comprising water.

Further aspects of the present invention include processes for preparing cannabinoid-containing compositions such as various food items, pharmaceuticals, topical compositions, and nutraceuticals comprising the nanoparticle dispersion as described herein or dilution or concentrate thereof. In some embodiments, these processes comprise preparing the cannabinoid nanoparticle dispersion as described herein; and mixing the cannabinoid nanoparticle dispersion or dilution or concentrate thereof with one or more ingredients of the food item, pharmaceutical, topical composition, or nutraceutical.

Still other aspects of the invention relate to methods of using pharmaceutical compositions comprising a cannabinoid-composition as described herein to treat one or more of the medical conditions in a subject in need thereof.

### BRIEF DESCRIPTION OF THE DRAWINGS

FIG. 1 shows the change in transmission signal of the CBD concentrate and dosed beverage (CBD OXIGEN water) at room temperature compared to tap water and the un-dosed beverage (OXIGEN water).

FIG. 2 shows the raw transmission signal of the CBD concentrate and CBD OXIGEN water at room temperature and 40° C. over the first 35 days of analysis.

FIG. 3. shows the raw transmission signal of the CBD concentrate and CBD OXIGEN water at room temperature and 40° C. over the first 35 days of analysis.

### DETAILED DESCRIPTION

The present disclosure provides stable, fast-acting cannabinoid-containing compositions comprising a cannabinoid and/or a cannabinoid analog that are compatible with aqueous formulations. For example, various compositions described herein include cannabinoid nanoparticle dispersions. These nanoparticle dispersions can provide for increased bioavailability, improved formulation stability, extended shelf life, enhanced optical clarity, and/or improved taste profile.

The present disclosure also provides for processes for preparing cannabinoid-containing compositions such as cannabinoid nanoparticle dispersions (e.g., cannabinoid nanoparticles in concentrate nanoemulsions). These processes beneficially provide for cannabinoid-containing compositions useful for food, beverage, pharmaceuticals, topical compositions, and nutraceuticals.

### Cannabinoid-Containing Compositions

As noted, various cannabinoid compositions of the present invention include cannabinoid nanoparticle dispersions. In various embodiments, the cannabinoid nanoparticle dispersions comprise:

a continuous phase comprising a carrier liquid component, and

nanoparticles at least partially dispersed in the carrier liquid component,

wherein the nanoparticles comprise a core material and a coating at least partially encapsulating the core material,

wherein the core material comprises a cannabinoid component comprising a cannabinoid and/or cannabinoid analog and the coating comprises an amphiphilic component, and

wherein the nanoparticles are characterized as having a mean particle size of about 60 nm or less, about 50 nm or less, about 40 nm or less, about 30 nm or less, about 25 nm or less, about 20 nm or less, or about 15 nm or less.

In some embodiments, the nanoparticles are characterized as having a mean particle size of from about 2 nm to about 60 nm, from about 5 nm to about 60 nm, from about 10 nm to about 60 nm, from about 15 nm to about 60 nm, from about 20 nm to about 60 nm, from about 2 nm to about 50 nm, from about 5 nm to about 50 nm, from about 10 nm to about 50 nm, from about 15 nm to about 50 nm, from about 20 nm to about 50 nm, from about 2 nm to about 40 nm, from about 5 nm to about 40 nm, from about 10 nm to about 40 nm, from about 15 nm to about 40 nm, from about 20 nm to about 40 nm, from about 2 nm to about 30 nm, from about 5 nm to about 30 nm, from about 10 nm to about 30 nm, from about 15 nm to about 30 nm, or from about 20 nm to about 30 nm. The mean particle size of the nanoparticles can be determined by measuring the particle size of a representative sample with a laser light scattering particle size analyzer known to those skilled in the art. Examples of particle size analyzers are the Malvern Zetasizer Nano ZS (dynamic particle size analyzer) and the Turbiscan Lab (Static Multiple Light Scattering).

As noted, the cannabinoid component comprises a cannabinoid and/or cannabinoid analog. The cannabinoid component can include oils, resins and molecules derived from the cannabis plant or modeled after the components found in the cannabis plant. This includes cannabinoids that are natural, semi-natural, synthetic or combinations thereof. The term “analog” refers to compound that is structurally related to naturally occurring cannabinoids, but whose chemical and biological properties may differ from naturally occurring cannabinoids. In the present context, analog or analogs refer compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid. According to one or more aspect, therefore, are provided compositions of cannabinoids and their analogs.

Specific examples of cannabinoids include delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), or combinations thereof. In various embodiments, the cannabinoid component comprises cannabidiol (CBD). In some embodiments, the cannabinoid component comprises tetrahydrocannabinol (THC).

In various embodiments, the cannabinoid nanoparticle dispersion has a concentration of the cannabinoid component that is about 0.1 wt. % or greater, about 0.25 wt. % or greater, about 0.5 wt. % or greater, about 0.75 wt. % or greater, about 1 wt. % or greater, about 1.5 wt. % or greater, or about 2 wt. % or greater. For example, the cannabinoid nanoparticle dispersion can have a concentration of the cannabinoid component that is from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1 wt. %, from about 0.5 wt. % to about 5 wt. %, from about 0.5 wt. % to about 4 wt. %, from about 0.5 wt. % to about 3 wt. %, from about 0.5 wt. % to about 2 wt. %, from about 0.5 wt. % to about 1 wt. %, from about 1 wt. % to about 5 wt. %, from about 1 wt. % to about 4 wt. %, from about 1 wt. % to about 3 wt. %, or from about 1 wt. % to about 2 wt. %.

The cannabinoid nanoparticle dispersion comprises a carrier liquid component. In various embodiments, the carrier liquid component comprises a polyol. In some embodiments, the carrier liquid component comprises a glycol. For example, in certain embodiments, the carrier liquid component comprises an alkylene glycol (e.g., propylene glycol).

Propylene glycol is clear, tasteless, and odorless. It is a generally recognized as safe (GRAS) ingredient for human consumption and can be used as a carrier for active ingredients (i.e., the cannabinoid or cannabinoid analog) and for increased absorption of the cannabinoid or cannabinoid analog in the body.

In some embodiments, the carrier liquid component comprises an oil, such as vegetable oil. In various embodiments, the carrier liquid component comprises glycerin, such as vegetable glycerin, which provides additional stability, protects the composition from oxidation, and adds flavor. The glycerin may be omitted in one or more embodiments.

In various embodiments, the carrier liquid component is an ingredient that is generally regarded as safe for human consumption.

The carrier liquid component typically constitutes a significant portion of the cannabinoid nanoparticle dispersion. In various embodiments, the cannabinoid nanoparticle dispersion has a concentration of the carrier liquid component that is about 40 wt. % or greater, about 50 wt. % or greater, about 60 wt. % or greater, about 70 wt. % or greater, about 80 wt. % or greater, or about 90 wt. % or greater. For example, the cannabinoid nanoparticle dispersion can have a concentration of the carrier liquid component that is from about 40 wt. % to about 95 wt. %, from about 40 wt. % to about 90 wt. %, from about 40 wt. % to about 80 wt. %, from about 40 wt. % to about 70 wt. %, from about 40 wt. % to about 60 wt. %, from about 40 wt. % to about 50 wt. %, from about 50 wt. % to about 95 wt. %, from about 50 wt. % to about 90 wt. %, from about 50 wt. % to about 80 wt. %, from about 50 wt. % to about 70 wt. %, from about 50 wt. % to about 60 wt. %, from about 60 wt. % to about 95 wt. %, from about 60 wt. % to about 90 wt. %, from about 60 wt. % to about 80 wt. %, from about 60 wt. % to about 70 wt. %, from about 70 wt. % to about 95 wt. %, from about 70 wt. % to about 90 wt. %, from about 70 wt. % to about 80 wt. %, from about 80 wt. % to about 95 wt. %, or from about 80 wt. % to about 90 wt. %.

As noted, the cannabinoid nanoparticle dispersion also comprises an amphiphilic component that functions as a coating that at least partially encapsulating the core material. In various embodiments, the amphiphilic component comprises an amphiphilic carbohydrate. In some embodiments, the amphiphilic component comprises an amphiphilic saccharide. For example, in certain embodiments, the amphiphilic component comprises an amphiphilic oligosaccharide and/or amphiphilic polysaccharide. In some embodiments, the amphiphilic component comprises a cyclic oligosaccharide such as cyclodextrin. In various embodiments, the amphiphilic component is an ingredient generally regarded as safe for human consumption.

In various embodiments, the cannabinoid nanoparticle dispersion has a concentration of the amphiphilic component that is about 0.1 wt. % or greater, about 0.25 wt. % or greater, about 0.5 wt. % or greater, about 0.75 wt. % or greater, about 1 wt. % or greater, about 2 wt. % or greater, about 3 wt. % or greater, about 4 wt. % or greater, about 5 wt. % or greater, or about 10 wt. % or greater. For example, the cannabinoid nanoparticle dispersion can have a concentration of the amphiphilic component that is from about 0.1 wt. % to about 10 wt. %, from about 0.1 wt. % to about 7.5 wt. %, from about 0.1 wt. % to about 5 wt. %, from about 0.1 wt. % to about 4 wt. %, from about 0.1 wt. % to about 3 wt. %, from about 0.5 wt. % to about 10 wt. %, from about 0.5 wt. % to about 7.5 wt. %, from about 0.5 wt. % to about 5 wt. %, from about 0.5 wt. % to about 4 wt. %, from about 0.5 wt. % to about 4 wt. %, from about 1 wt. % to about 10 wt. %, from about 1 wt. % to about 7.5 wt. %, from about 1 wt. % to about 5 wt. %, from about 1 wt. % to about 4 wt. %, or from about 1 wt. % to about 3 wt. %.

In various embodiments, the cannabinoid nanoparticle dispersion further comprises water. When water is present, the nanoparticles can be at least partially dispersed in the continuous phase comprising carrier liquid component and water. In various embodiments, the cannabinoid nanoparticle dispersion is an oil in water (0/W) emulsion (or nanoemulsion).

Water (e.g., deionized water or distilled water) provides compatibility with aqueous formulations. Water in the oil phase solution is present during the creation of the cannabinoid or cannabinoid analog nanoparticles. A different type of water, other than deionized water, may be used. For example, the water may be clustered water or a different type of water. Clustered water creates smaller water molecules to reduce the surface tension and allow increased absorption when mixing with the nanoparticles.

In some embodiments, the cannabinoid nanoparticle dispersion has a concentration of water that is about 1 wt. % or greater, about 2 wt. % or greater, about 3 wt. % or greater, about 4 wt. % or greater, about 5 wt. % or greater, or about 10 wt. % or greater. In certain embodiments, the cannabinoid nanoparticle dispersion has a concentration of water that is from about 1 wt. % to about 20 wt. %, from about 1 wt. % to about 15 wt. %, from about 1 wt. % to about 12 wt. %, from about 1 wt. % to about 10 wt. %, from about 5 wt. % to about 20 wt. %, from about 5 wt. % to about 15 wt. %, from about 5 wt. % to about 12 wt. %, or from about 5 wt. % to about 10 wt. %.

In various embodiments, the cannabinoid nanoparticle dispersion is optically transparent, substantially optically transparent, or translucent. In some embodiments, the cannabinoid nanoparticle dispersion exhibits an approximately neutral charge.

Other cannabinoid-containing compositions of the present invention include for example a drink or beverage composition comprising a dilution (e.g., aqueous dilution) of the cannabinoid nanoparticle dispersion as described herein. Further cannabinoid-containing compositions of the present invention include various food items, pharmaceuticals, topical compositions, and nutraceuticals comprising the cannabinoid nanoparticle dispersion as described herein or dilution or concentrate thereof.

For example, the pharmaceutical can contain a drug or therapy that has a pharmacological effect in the composition. Examples of such drugs include, but are not limited to: opioids, steroids, chemotherapeutic agents, immunosuppressive agents, immunostimulatory, antipyretic, cytokines, cytotoxic agents, nucleolytic compounds, radioactive isotopes, enzymes, antibiotics, growth factors, protease inhibitors, analgesics, antidepressants, stimulants, antibodies, beta-blockers, anti-inflammatory agents, central nervous system depressants, etc.

Nutraceuticals can include functional or nutritional ingredients. Examples of such ingredients include, but are not limited to: caffeine, chamomile, b-vitamins, protein, omega-3 fatty acids, creatine, L-arginine, St. John's wort, horny goat weed, valerian root, phenylalanine, guarana, taurine, melatonin, turmeric, vitamins (A, B, E, C, etc.), echinacea, minerals (calcium, sodium, magnesium, zinc etc.), etc.

The cannabinoid-containing compositions (e.g., the cannabinoid nanoparticle dispersion as described herein) can further comprise other ingredients such as other ingredients that are generally accepted as safe for human consumption. For example, the compositions (e.g., the cannabinoid nanoparticle dispersion) can comprise a fat.

In some embodiments, the compositions (e.g., the cannabinoid nanoparticle dispersion) further comprise a sugar, sugar alcohol, and/or sugar derivative. Specific examples include dextrose, trehalose, sucrose, sucrose esters, among others.

In certain embodiments, the compositions (e.g., the cannabinoid nanoparticle dispersion) further comprise a sweetener. The sweetener may be a zero calorie sweetener, such as a steviol glycoside. For example, the sweetener may be a stevioside or a rebaudioside. In one example, the sweetener is one or more, or a combination thereof, of the following: rebaudioside A, rebaudioside B, rebaudioside C, rebaudioside D, rebaudioside E, or dulcoside A. Other types of sweeteners may be used. In one or more embodiments, the sweetener may be omitted from the composition.

In certain embodiments, the compositions (e.g., the cannabinoid nanoparticle dispersion) further comprise a triglyceride (e.g., medium chain triglycerides (MCT)). MCT oil provides liposome encapsulation. In some embodiments, a different ingredient may be used to provide liposome encapsulation. In one or more embodiments, the MCT oil or other ingredient for liposome encapsulation may be omitted. For example, in some embodiments, the compositions (e.g., the cannabinoid nanoparticle dispersion) are free or essentially free (e.g., less than 0.1 wt. % or even less than 0.01 wt. %) of triglycerides. In certain embodiments, the compositions (e.g., the cannabinoid nanoparticle dispersion) are free or essentially free of medium chain triglyceride (MCT) oil.

In various embodiments, the compositions (e.g., the cannabinoid nanoparticle dispersion) further comprise a carboxylic acid, such as a polycarboxylic acid (e.g., citric acid).

In some embodiments, the compositions (e.g., the cannabinoid nanoparticle dispersion) further comprise a further comprises a preservative and/or antioxidant. The antioxidant can include inner Aloe leaf or another antioxidant. The inner Aloe leaf protects the product from oxidation, increasing shelf stability. It is believed the inner Aloe leaf increases absorption of the cannabinoid or cannabinoid analog in the body.

In certain embodiments, the compositions (e.g., the cannabinoid nanoparticle dispersion) further comprise a flavoring to enhance the taste profile of the composition.

In various embodiments, all components of the cannabinoid-containing compositions (e.g., the cannabinoid nanoparticle dispersion) are ingredients that are generally regarded as safe for human consumption.

### Processes for Preparing Cannabinoid-Containing Compositions

As noted, various aspects of the present invention relate to processes for preparing cannabinoid-containing compositions. The processes include those for preparing cannabinoid nanoparticle dispersion including those described herein. In some embodiments, the processes comprise:

exposing a mixture comprising a carrier liquid component, an amphiphilic component, and a cannabinoid component comprising a cannabinoid and/or cannabinoid analog to electromagnetic radiation in a heating zone to form the cannabinoid nanoparticle dispersion comprising nanoparticles at least partially dispersed in a continuous phase comprising at least a portion of the carrier liquid component,

wherein the nanoparticles comprise a core material and a coating at least partially encapsulating the core material, wherein the core material comprises at least a portion of the cannabinoid component and the coating comprises at least a portion of the amphiphilic component, and wherein the nanoparticles are characterized as having a mean particle size of about 60 nm or less, about 50 nm or less, about 40 nm or less, about 30 nm or less, about 25 nm or less, about 20 nm or less, or about 15 nm or less.

As noted herein, in some embodiments, the nanoparticles are characterized as having a mean particle size of from about 2 nm to about 60 nm, from about 5 nm to about 60 nm, from about 10 nm to about 60 nm, from about 15 nm to about 60 nm, from about 20 nm to about 60 nm, from about 2 nm to about 50 nm, from about 5 nm to about 50 nm, from about 10 nm to about 50 nm, from about 15 nm to about 50 nm, from about 20 nm to about 50 nm, from about 2 nm to about 40 nm, from about 5 nm to about 40 nm, from about 10 nm to about 40 nm, from about 15 nm to about 40 nm, from about 20 nm to about 40 nm, from about 2 nm to about 30 nm, from about 5 nm to about 30 nm, from about 10 nm to about 30 nm, from about 15 nm to about 30 nm, or from about 20 nm to about 30 nm. The mean particle size of the nanoparticles can be determined by measuring the particle size of a representative sample with a laser light scattering particle size analyzer known to those skilled in the art. Examples of dynamic particle size analyzer is a Malvern Zetasizer Nano ZS (dynamic particle size analyzer) and Turbiscan Lab (Static Multiple Light Scattering).

In one or more embodiments, electromagnetic radiation is applied to the mixture (e.g., liquid mixture) containing the cannabinoid and/or cannabinoid analog with a particle size greater than about 25 nm, such as greater than about 100 nm, or greater than about 200 nm, or greater than about 300 nm, or greater than 400 nm, or greater than 500 nm, for example, from about 100 nm to about 500 nm, or from about 100 nm to about 400 nm, or from about 200 nm to about 300n m. In one example, the electromagnetic radiation is suitable for heating (e.g., dielectric heating) the mixture and facilitating reduction of the size of the particles comprising the cannabinoid and/or cannabinoid analog. For example, the size of the particles comprising the cannabinoid and/or cannabinoid analog can be reduced to less than about 50 nm, or less than about 40 nm, or less than about 30 nm, or less than about 25 nm, for example, from about 1 nm to about 50 nm, or from about 1 nm to about 40 nm, or from about 1 nm to about 30 nm, or from about 1 nm to about 25 nm, or from about 5 nm to about 50 nm, or from about 5 nm to about 40 nm, or from about 5 nm to about 30 nm, or from about 5 nm to about 25 nm, or from about from about 10 nm to about 50 nm, or from about 10 nm to about 40 nm, or from about 10 nm to about 30 nm, or from about 10 nm to about 25 nm, or from about 15 nm to about 50 nm, or from about 15 nm to about 40 nm, or from about 15 nm to about 30 nm, or from about 15 nm to about 25 nm, from about 20 nm to about 50 nm, or from about 20 nm to about 40 nm, or from about 20 nm to about 30 nm, or from about 20 nm to about 25 nm.

In one or more embodiments, the electromagnetic radiation applied to the mixture comprising a carrier liquid component, an amphiphilic component, and a cannabinoid component comprising a cannabinoid and/or cannabinoid analog comprises microwave radiation. Microwave radiation has a frequency in the microwave spectrum (i.e., from about 300 MHz to about 300 GHz). In one or more examples, the applied electromagnetic radiation may have a frequency from about 500 MHz to about 5 GHz, or from about 700 MHz to about 4 GHz, or from about 900 MHz to about 3 GHz, from about 1 GHz to about 3 GHz, or from about 1.5 GHz to about 2.5 GHz. The frequency of the applied microwave radiation may be suitable for inducing polar molecules in the mixture to rotate and produce thermal energy through dielectric heating. Water, fat, and other substances in the mixture absorb energy from the microwave radiation. The applied microwave radiation is also suitable to reduce the particle size of the cannabinoid or cannabinoid analog particulate ingredient to the size described in the above paragraph.

In one or more embodiments, the use of microwave radiation facilitates dissolution of the cannabinoid or cannabinoid analog in the liquid solvent to form a cannabinoid or cannabinoid analog solution. During or after application of microwave radiation, the mixture/solution may be agitated (e.g., stirred or mixed) to further facilitate dissolution of the cannabinoid or cannabinoid analog (i.e., solute) in the liquid solvent (e.g., water). This cannabinoid or cannabinoid analog solution may be translucent or substantially transparent (i.e., clear). The cannabinoid or cannabinoid analog in the liquid solution may also be essentially tasteless and/or odorless.

In some embodiments, the microwave radiation is generated by an inverter microwave oven. An inverter microwave oven provides a constant flow of vortex cyclonic frequency that gives accurate, true power levels (e.g., not small bursts of microwaves 60% of the time as in conventional microwave ovens). In an inverter microwave oven, the power transformer is replaced by a circuit board, which converts the 60Hz incoming line frequency to a variable rate of 20 KHz to 45 KHz. A relatively small transformer is then required to increase the voltage to the level required by a magnetron. By varying the pulse width, the output power can be linearly controlled for more precise manufacturing and reproducibility.

It has been discovered that microwave radiation, particularly microwave radiation generated by an inverter microwave oven provides for rapid, uniform heating resulting in nanoparticles comprising the cannabinoid component having reduce particle size. Microwave radiation also provides for desired microbial and pathogen lethality without altering or degrading the overall quality of the cannabinoid and other ingredients. For example, bacteria reported to be inactivated by microwave heating includes Bacillus cereus, Campylobacter jejuni, Clostridium perfringens, Pathogenic Escherichia coli, Enterococcus, Listeria monocytogenes, and Staphylococcus aureus. Uniform heating also prevents nutrient loss of heat sensitive and highly volatile components as opposed to conventional methods that use surface heating and lead to degradation on the outer portion and neglect the center.

Accordingly, in various embodiments, the microwave radiation is generated by electrical power and the electrical power is linearly controlled. In certain embodiments, the electrical power is not pulse controlled. In some embodiments, during exposure, the microwave radiation is continuous. Also, in various embodiments, during exposure, the microwave radiation is not intermittent or pulsed. In certain embodiments, during exposure, the frequency of the microwave radiation is 300 MHz or greater, 500 MHz or greater, or 1 GHz or greater.

Further processes of the present invention provide essentially for 4-D printing of nanoparticles comprising a cannabinoid component.

In various embodiments, the process further comprises generating the electromagnetic radiation.

In some embodiments, the mixture is heated in the heating zone to a temperature of from about 75° C. to about 100° C., from about 75° C. to about 95° C., or from about 80° C. to about 95° C. by exposure to the electromagnetic radiation (e.g., microwave radiation). In various embodiments, the mixture prior to exposure to the electromagnetic radiation is at room temperature (e.g., 20° C.-25° C.).

In various embodiments, the mixture further comprises a magnetic material. In some embodiments, the magnetic material comprises ferrite. It has been discovered that a magnetic material in the mixture functions as an additional focus point that attracts electromagnetic waves, particularly microwaves into the mixture, reflects the waves dissipating into the mixture, and enhances volumetric heating. Including a magnetic material has been found to increase the rate of processing the mixture and forming nanoparticles. In certain embodiments, the magnetic material is coated (e.g., polymer coating such as PTFE).

In various embodiments, the heating zone comprise a multimode oven cavity resonator designed to resonate the microwaves emitted from the magnetron and reflect the microwaves. This adds convection heating to surface material as waves bounce off the cavity walls and are attracted to the magnet material that may be present in the mixture.

In various embodiments, the mixture is held within a vessel at least partially constructed of a heat insulating material. In some embodiments, the mixture is held within a vessel at least partially constructed of a microwave conducting material. For example, the heat insulating/microwave conducting material can comprise glass or ceramic.

In some embodiments, the vessel is cylindrical in shape. A cylindrical shape can further improve uniformity of heating of the mixture in the heating zone.

Also, in certain embodiments, the process further comprises rotating the vessel during exposure to the electromagnetic radiation. Rotating the vessel during exposure can enhance the uniformity of heating of the mixture.

In various embodiments, the process further comprises stirring or mixing the mixture before or during exposure to the electromagnetic radiation. In some embodiments, the process also comprises stirring or mixing the cannabinoid nanoparticle dispersion after formation.

In various embodiments, the process can further comprise various downstream steps after exposure to the electromagnetic radiation. For example, additional components such as sugars, sweeteners, and flavorings can be added to the cannabinoid nanoparticle dispersion. Also, in some embodiments, the cannabinoid nanoparticle dispersion can be subjected to a separation operation such as filtration to remove unwanted fractions.

In various embodiments, the cannabinoid nanoparticle dispersion produced by the processes of the present invention is optically transparent, substantially optically transparent, or translucent. In some embodiments, the cannabinoid nanoparticle dispersion produced by the processes of the present invention exhibits an approximately neutral charge.

The processes of the present invention can include one or features of the various cannabinoid-containing compositions described herein. For example, the cannabinoid component comprises a cannabinoid and/or cannabinoid analog. The cannabinoid component can include oils, resins and molecules derived from the cannabis plant or modeled after the components found in the cannabis plant. This includes cannabinoids that are natural, semi-natural, synthetic or combinations thereof The term “analog” refers to compound that is structurally related to naturally occurring cannabinoids, but whose chemical and biological properties may differ from naturally occurring cannabinoids. In the present context, analog or analogs refer compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid. According to one or more embodiments, therefore, are provided compositions of cannabinoids and their analogs.

Specific examples of cannabinoids include delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), or combinations thereof In various embodiments, the cannabinoid component comprises cannabidiol (CBD). In some embodiments, the cannabinoid component comprises tetrahydrocannabinol (THC). When delta-9-tetrahydrocannabinolic acid (THCa) is present in the mixture, various processes of the present invention have been found to convert this compound to THC.

In various embodiments, the mixture and/or cannabinoid nanoparticle dispersion has a concentration of the cannabinoid component that is about 0.1 wt. % or greater, about 0.25 wt. % or greater, about 0.5 wt. % or greater, about 0.75 wt. % or greater, about 1 wt. % or greater, about 1.5 wt. % or greater, or about 2 wt. % or greater. For example, the mixture and/or cannabinoid nanoparticle dispersion can have a concentration of the cannabinoid component that is from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1 wt. %, from about 0.5 wt. % to about 5 wt. %, from about 0.5 wt. % to about 4 wt. %, from about 0.5 wt. % to about 3 wt. %, from about 0.5 wt. % to about 2 wt. %, from about 0.5 wt. % to about 1 wt. %, from about 1 wt. % to about 5 wt. %, from about 1 wt. % to about 4 wt. %, from about 1 wt. % to about 3 wt. %, or from about 1 wt. % to about 2 wt. %.

The mixture and cannabinoid nanoparticle dispersion comprises a carrier liquid component. In various embodiments, the carrier liquid component comprises a polyol. In some embodiments, the carrier liquid component comprises a glycol. For example, in certain embodiments, the carrier liquid component comprises an alkylene glycol (e.g., propylene glycol).

Propylene glycol is clear, tasteless, and odorless. It is a generally recognized as safe (GRAS) ingredient for human consumption and can be used as a carrier for active ingredients (i.e., the cannabinoid or cannabinoid analog) and for increased absorption of the cannabinoid or cannabinoid analog in the body.

In some embodiments, the carrier liquid component comprises an oil, such as vegetable oil. In various embodiments, the carrier liquid component comprises glycerin, such as vegetable glycerin, which provides additional stability, protects the composition from oxidation, and adds flavor. The glycerin may be omitted in one or more embodiments.

In various embodiments, wherein the carrier liquid component is an ingredient that is generally regarded as safe for human consumption.

The carrier liquid component typically constitutes a significant portion of the mixture and/or cannabinoid nanoparticle dispersion. In various embodiments, the mixture and/or cannabinoid nanoparticle dispersion has a concentration of the carrier liquid component that is about 40 wt. % or greater, about 50 wt. % or greater, about 60 wt. % or greater, about 70 wt. % or greater, about 80 wt. % or greater, or about 90 wt. % or greater. For example, the mixture and/or cannabinoid nanoparticle dispersion can have a concentration of the carrier liquid component that is from about 40 wt. % to about 95 wt. %, from about 40 wt. % to about 90 wt. %, from about 40 wt. % to about 80 wt. %, from about 40 wt. % to about 70 wt. %, from about 40 wt. % to about 60 wt. %, from about 40 wt. % to about 50 wt. %, from about 50 wt. % to about 95 wt. %, from about 50 wt. % to about 90 wt. %, from about 50 wt. % to about 80 wt. %, from about 50 wt. % to about 70 wt. %, from about 50 wt. % to about 60 wt. %, from about 60 wt. % to about 95 wt. %, from about 60 wt. % to about 90 wt. %, from about 60 wt. % to about 80 wt. %, from about 60 wt. % to about 70 wt. %, from about 70 wt. % to about 95 wt. %, from about 70 wt. % to about 90 wt. %, from about 70 wt. % to about 80 wt. %, from about 80 wt. % to about 95 wt. %, or from about 80 wt. % to about 90 wt. %.

As noted, the mixture and cannabinoid nanoparticle dispersion also comprise an amphiphilic component that functions as a coating that at least partially encapsulating the core material. In various embodiments, the amphiphilic component comprises an amphiphilic carbohydrate. In some embodiments, the amphiphilic component comprises an amphiphilic saccharide. For example, in certain embodiments, the amphiphilic component comprises an amphiphilic oligosaccharide and/or amphiphilic polysaccharide. In some embodiments, the amphiphilic component comprises a cyclic oligosaccharide such as cyclodextrin. In various embodiments, the amphiphilic component is an ingredient generally regarded as safe for human consumption.

In various embodiments, the mixture and cannabinoid nanoparticle dispersion has a concentration of the amphiphilic component that is about 0.1 wt. % or greater, about 0.25 wt. % or greater, about 0.5 wt. % or greater, about 0.75 wt. % or greater, about 1 wt. % or greater, about 2 wt. % or greater, about 3 wt. % or greater, about 4 wt. % or greater, about 5 wt. % or greater, or about 10 wt. % or greater. For example, the mixture and/or cannabinoid nanoparticle dispersion can have a concentration of the amphiphilic component that is from about 0.1 wt. % to about 10 wt. %, from about 0.1 wt. % to about 7.5 wt. %, from about 0.1 wt. % to about 5 wt. %, from about 0.1 wt. % to about 4 wt. %, from about 0.1 wt. % to about 3 wt. %, from about 0.5 wt. % to about 10 wt. %, from about 0.5 wt. % to about 7.5 wt. %, from about 0.5 wt. % to about 5 wt. %, from about 0.5 wt. % to about 4 wt. %, from about 0.5 wt. % to about 4 wt. %, from about 1 wt. % to about 10 wt. %, from about 1 wt. % to about 7.5 wt. %, from about 1 wt. % to about 5 wt. %, from about 1 wt. % to about 4 wt. %, or from about 1 wt. % to about 3 wt. %.

In various embodiments, the mixture and/or cannabinoid nanoparticle dispersion further comprises water. When water is present, the nanoparticles can be at least partially dispersed in the continuous phase comprising carrier liquid component and water. In various embodiments, the cannabinoid nanoparticle dispersion is an oil in water (O/W) emulsion (or nanoemulsion).

Water (e.g., deionized water or distilled water) provides compatibility with aqueous formulations. Water in the oil phase solution is present during the creation of the cannabinoid or cannabinoid analog nanoparticles. A different type of water, other than deionized water, may be used. For example, the water may be clustered water or a different type of water. Clustered water creates smaller water molecules to reduce the surface tension and allow increased absorption when mixing with the nanoparticles.

In some embodiments, the mixture and/or cannabinoid nanoparticle dispersion has a concentration of water that is about 1 wt. % or greater, about 2 wt. % or greater, about 3 wt. % or greater, about 4 wt. % or greater, about 5 wt. % or greater, or about 10 wt. % or greater. In certain embodiments, the mixture and/or cannabinoid nanoparticle dispersion has a concentration of water that is from about 1 wt. % to about 20 wt. %, from about 1 wt. % to about 15 wt. %, from about 1 wt. % to about 12 wt. %, from about 1 wt. % to about 10 wt. %, from about 5 wt. % to about 20 wt. %, from about 5 wt. % to about 15 wt. %, from about 5 wt. % to about 12 wt. %, or from about 5 wt. % to about 10 wt. %.

The mixture and/or cannabinoid nanoparticle dispersion can further comprise other ingredients such as other ingredients that are generally accepted as safe for human consumption. For example, the compositions (e.g., the cannabinoid nanoparticle dispersion) can comprise a fat.

In some embodiments, the mixture and/or cannabinoid nanoparticle dispersion further comprise a sugar, sugar alcohol, and/or sugar derivative. Specific examples include dextrose, trehalose, sucrose, sucrose esters, among others.

In certain embodiments, the mixture and/or cannabinoid nanoparticle dispersion further comprise a sweetener. The sweetener may be a zero calorie sweetener, such as a steviol glycoside. For example, the sweetener may be a stevioside or a rebaudioside. In one example, the sweetener is one or more, or a combination thereof, of the following: rebaudioside A, rebaudioside B, rebaudioside C, rebaudioside D, rebaudioside E, or dulcoside A. Other types of sweeteners may be used. In one or more embodiments, the sweetener may be omitted from the composition.

In certain embodiments, the mixture and/or cannabinoid nanoparticle dispersion further comprise a triglyceride (e.g., medium chain triglycerides (MCT)). MCT oil provides liposome encapsulation. In some embodiments, a different ingredient may be used to provide liposome encapsulation. In one or more embodiments, the MCT oil or other ingredient for liposome encapsulation may be omitted. For example, in some embodiments, the mixture and/or cannabinoid nanoparticle dispersion is free or essentially free (e.g., less than 0.1 wt. % or even less than 0.01 wt. %) of triglycerides. In certain embodiments, the mixture and/or cannabinoid nanoparticle dispersion is free or essentially free of medium chain triglyceride (MCT) oil.

In various embodiments, the mixture and/or cannabinoid nanoparticle dispersion further comprise a carboxylic acid, such as a polycarboxylic acid (e.g., citric acid).

In some embodiments, the mixture and/or cannabinoid nanoparticle dispersion further comprise a further comprises a preservative and/or antioxidant. The antioxidant can include inner Aloe leaf or another antioxidant. The inner Aloe leaf protects the product from oxidation, increasing shelf stability. It is believed the inner Aloe leaf increases absorption of the cannabinoid or cannabinoid analog in the body.

In certain embodiments, the mixture and/or cannabinoid nanoparticle dispersion further comprise a flavoring to enhance the taste profile of the composition.

In various embodiments, all components of the mixture and/or cannabinoid nanoparticle dispersion are ingredients that are generally regarded as safe for human consumption.

Aspects of the present invention also include processes for preparing other cannabinoid-containing compositions such as a drink or beverage composition comprising a dilution (e.g., aqueous dilution) of the nanoparticle dispersion as described herein. In some embodiments, these processes comprise preparing the cannabinoid nanoparticle dispersion as described herein; and diluting the cannabinoid nanoparticle dispersion with a liquid comprising water.

Further aspects of the present invention include processes for preparing cannabinoid-containing compositions such as various food items, pharmaceuticals, topical compositions, and nutraceuticals comprising the nanoparticle dispersion as described herein or dilution or concentrate thereof. In some embodiments, these processes comprise preparing the cannabinoid nanoparticle dispersion as described herein; and mixing the cannabinoid nanoparticle dispersion or dilution or concentrate thereof with one or more ingredients of the food item, pharmaceutical, topical composition, or nutraceutical.

For example, the pharmaceutical can contain a drug or therapy that has a pharmacological effect in the composition. Examples of such drugs include, but are not limited to: opioids, steroids, chemotherapeutic agents, immunosuppressive agents, immunostimulatory, antipyretic, cytokines, cytotoxic agents, nucleolytic compounds, radioactive isotopes, enzymes, antibiotics, growth factors, protease inhibitors, analgesics, antidepressants, stimulants, antibodies, beta-blockers, anti-inflammatory agents, central nervous system depressants, etc.

Nutraceuticals can include functional or nutritional ingredients. Examples of such ingredients include, but are not limited to: caffeine, chamomile, b-vitamins, protein, omega-3 fatty acids, creatine, L-arginine, St. John's wort, horny goat weed, valerian root, phenylalanine, guarana, taurine, melatonin, turmeric, vitamins (A, B, E, C, etc.), echinacea, minerals (calcium, sodium, magnesium, zinc etc.), etc.

### Methods of Use

The present invention also relates to various methods of using the cannabinoid-compositions described herein to treat a medical condition of a subject in need thereof. Cannabinoid components possess therapeutic properties including analgesia, ocular hypotension, and antiemesis. Cannabinoids-based medications can be used for treatment of a wide range of medical conditions, including neuropathic pain, pain related to cancer and trauma, spasticity associated with multiple sclerosis, fibromyalgia, gastrointestinal, metabolic, neurological, circulatory, soft tissue, musculoskeletal, chronic or acute pain, nausea, decreased appetite, skin disorders, sexual dysfunction, glaucoma, AIDS wasting, neuropathic pain, treatment of spasticity associated with multiple sclerosis, fibromyalgia, chemotherapy-induced nausea, allergies, inflammation, infection, epilepsy, depression, migraine, bipolar disorders, anxiety disorder, dependency and withdrawal. Cannabinoids-based medications can be used to alleviate, or relief symptoms or side effects associated with anti-retroviral therapy, chemotherapy and radiation therapy.

Further, cannabinoids can reduce ACE2 expression and pro-inflammatory cytokine production to fight lung inflammation. Also, cannabinoids can possess antiviral activity. Thus, cannabinoids can be used to treat viral infections or symptoms such as lung inflammation caused by viral infections (e.g., caused by various coronaviruses such as SARS-CoV-2, SARS-CoV, MERS-CoV and other coronaviruses and influenza).

The cannabinoid-containing compositions of the present invention can exhibit enhanced bioavailability of the cannabinoid active while reducing or eliminating undesirable oils that may affect liver health. Thus, these compositions are particularly suited for treating medical conditions. Further, in some embodiments, as a result of the enhanced bioavailability, lower concentrations of the cannabinoid may be needed when using pharmaceutical compositions comprising the cannabinoid-containing compositions as described herein.

Accordingly, in various embodiments, a pharmaceutical composition comprising a cannabinoid-composition as described herein is administered to a subject in need thereof to treat one or more of the medical conditions noted above. The pharmaceutical composition can also include of be co-administered with an additional drug as described herein.

A pharmaceutical composition for oral administration can be formulated using pharmaceutically acceptable carriers known in the art in dosages suitable for oral administration. Such carriers enable the pharmaceutical compositions to be formulated as tablets, pills, dragees, capsules, liquids, gels, syrups, slurries, suspensions, and the like, for ingestion by the subject. Further details on techniques for formulation and administration can be found in the latest edition of REMINGTON'S PHARMACEUTICAL SCIENCES (Mack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

### Embodiments

For further illustration, additional non-limiting embodiments of the present disclosure are set forth below.

Embodiment 1 is a process for preparing a cannabinoid nanoparticle dispersion, the process comprising:

exposing a mixture comprising a carrier liquid component, an amphiphilic component, and a cannabinoid component comprising a cannabinoid and/or cannabinoid analog to electromagnetic radiation in a heating zone to form the cannabinoid nanoparticle dispersion comprising nanoparticles at least partially dispersed in a continuous phase comprising at least a portion of the carrier liquid component,

wherein the nanoparticles comprise a core material and a coating at least partially encapsulating the core material,

wherein the core material comprises at least a portion of the cannabinoid component and the coating comprises at least a portion of the amphiphilic component, and

wherein the nanoparticles are characterized as having a mean particle size of about 60 nm or less, about 50 nm or less, about 40 nm or less, about 30 nm or less, about 25 nm or less, about 20 nm or less, or about 15 nm or less.

Embodiment 2. The process of Embodiment 1, wherein the nanoparticles are characterized as having a mean particle size of from about 2 nm to about 60 nm, from about 5 nm to about 60 nm, from about 10 nm to about 60 nm, from about 15 nm to about 60 nm, from about 20 nm to about 60 nm, from about 2 nm to about 50 nm, from about 5 nm to about 50 nm, from about 10 nm to about 50 nm, from about 15 nm to about 50 nm, from about 20 nm to about 50 nm, from about 2 nm to about 40 nm, from about 5 nm to about 40 nm, from about 10 nm to about 40 nm, from about 15 nm to about 40 nm, from about 20 nm to about 40 nm, from about 2 nm to about 30 nm, from about 5 nm to about 30 nm, from about 10 nm to about 30 nm, from about 15 nm to about 30 nm, or from about 20 nm to about 30 nm.

Embodiment 3. The process of Embodiment 1 or 2, further comprising generating the electromagnetic radiation.

Embodiment 4. The process of any one of Embodiments 1 to 3, wherein the electromagnetic radiation comprises microwave radiation.

Embodiment 5. The process of Embodiment 3 or 4, wherein the microwave radiation is generated by electrical power and the electrical power is linearly controlled.

Embodiment 6. The process of Embodiment 5, wherein the electrical power is not pulse controlled.

Embodiment 7. The process of any one of Embodiments 3 to 6, wherein the microwave radiation is generated by an inverter microwave oven.

Embodiment 8. The process of any one of Embodiments 3 to 7, wherein the microwave radiation has a frequency that is 500 MHz to about 5 GHz, or from about 700 MHz to about 4 GHz, from about 900 MHz to about 3 GHz, from about 1 GHz to about 3 GHz, or from about 1.5 GHz to about 2.5 GHz.

Embodiment 9. The process of any one of Embodiments 3 to 8, wherein during exposure, the microwave radiation is continuous.

Embodiment 10. The process of any one of Embodiments 3 to 9, wherein during exposure, the microwave radiation is not intermittent or pulsed.

Embodiment 11. The process of any one of Embodiments 3 to 10, wherein during exposure, the frequency of the microwave radiation is 300 MHz or greater, 500 MHz or greater, or 1 GHz or greater.

Embodiment 12. The process of any one of Embodiments 1 to 11, wherein the mixture is heated to a temperature of from about 75° C. to about 100° C., from about 75° C. to about 95° C., or from about 80° C. to about 95° C. by exposure to the electromagnetic radiation.

Embodiment 13. The process of any one of Embodiments 1 to 12, wherein the cannabinoid component comprises at least one compound selected from the group consisting of delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), and combinations thereof.

Embodiment 14. The process of any one of Embodiments 1 to 13, wherein the cannabinoid component comprises cannabidiol (CBD).

Embodiment 15. The process of any one of Embodiments 1 to 14, wherein the cannabinoid component comprises tetrahydrocannabinol (THC).

Embodiment 16. The process of any one of Embodiments 1 to 15, wherein the mixture and/or cannabinoid nanoparticle dispersion has a concentration of the cannabinoid component that is about 0.1 wt. % or greater, about 0.25 wt. % or greater, about 0.5 wt. % or greater, about 0.75 wt. % or greater, about 1 wt. % or greater, about 1.5 wt. % or greater, or about 2 wt. % or greater.

Embodiment 17. The process of any one of Embodiments 1 to 16, wherein the mixture and/or cannabinoid nanoparticle dispersion has a concentration of the cannabinoid component that is from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1 wt. %, from about 0.5 wt. % to about 5 wt. %, from about 0.5 wt. % to about 4 wt. %, from about 0.5 wt. % to about 3 wt. %, from about 0.5 wt. % to about 2 wt. %, from about 0.5 wt. % to about 1 wt. %, from about 1 wt. % to about 5 wt. %, from about 1 wt. % to about 4 wt. %, from about 1 wt. % to about 3 wt. %, or from about 1 wt. % to about 2 wt. %.

Embodiment 18. The process of any one of Embodiments 1 to 17, wherein the carrier liquid component comprises a polyol.

Embodiment 19. The process of any one of Embodiments 1 to 18, wherein the carrier liquid component comprises a glycol.

Embodiment 20. The process of any one of Embodiments 1 to 19, wherein the carrier liquid component comprises an alkylene glycol.

Embodiment 21. The process of any one of Embodiments 1 to 20, wherein the carrier liquid component comprises a propylene glycol.

Embodiment 22. The process of any one of Embodiments 1 to 21, wherein the carrier liquid component comprises a vegetable oil.

Embodiment 23. The process of any one of Embodiments 1 to 22, wherein the mixture and/or cannabinoid nanoparticle dispersion has a concentration of the carrier liquid component that is about 40 wt. % or greater, about 50 wt. % or greater, about 60 wt. % or greater, about 70 wt. % or greater, about 80 wt. % or greater, or about 90 wt. % or greater.

Embodiment 24. The process of any one of Embodiments 1 to 23, wherein the mixture and/or cannabinoid nanoparticle dispersion has a concentration of the carrier liquid component that is from about 40 wt. % to about 95 wt. %, from about 40 wt. % to about 90 wt. %, from about 40 wt. % to about 80 wt. %, from about 40 wt. % to about 70 wt. %, from about 40 wt. % to about 60 wt. %, from about 40 wt. % to about 50 wt. %, from about 50 wt. % to about 95 wt. %, from about 50 wt. % to about 90 wt. %, from about 50 wt. % to about 80 wt. %, from about 50 wt. % to about 70 wt. %, from about 50 wt. % to about 60 wt. %, from about 60 wt. % to about 95 wt. %, from about 60 wt. % to about 90 wt. %, from about 60 wt. % to about 80 wt. %, from about 60 wt. % to about 70 wt. %, from about 70 wt. % to about 95 wt. %, from about 70 wt. % to about 90 wt. %, from about 70 wt. % to about 80 wt. %, from about 80 wt. % to about 95 wt. %, or from about 80 wt. % to about 90 wt. %.

Embodiment 25. The process of any one of Embodiments 1 to 24, wherein the amphiphilic component comprises an amphiphilic carbohydrate.

Embodiment 26. The process of any one of Embodiments 1 to 25, wherein the amphiphilic component comprises an amphiphilic saccharide.

Embodiment 27. The process of any one of Embodiments 1 to 26, wherein the amphiphilic component comprises an amphiphilic oligosaccharide and/or amphiphilic polysaccharide.

Embodiment 28. The process of any one of Embodiments 1 to 27, wherein the amphiphilic component comprises a cyclic oligosaccharide.

Embodiment 29. The process of any one of Embodiments 1 to 28, wherein the amphiphilic component comprises cyclodextrin.

Embodiment 30. The process of any one of Embodiments 1 to 29, wherein the mixture and/or cannabinoid nanoparticle dispersion has a concentration of the amphiphilic component that is about 0.1 wt. % or greater, about 0.25 wt. % or greater, about 0.5 wt. % or greater, about 0.75 wt. % or greater, about 1 wt. % or greater, about 2 wt. % or greater, about 3 wt. % or greater, about 4 wt. % or greater, about 5 wt. % or greater, or about 10 wt. % or greater.

Embodiment 31. The process of any one of Embodiments 1 to 30, wherein the mixture and/or cannabinoid nanoparticle dispersion has a concentration of the amphiphilic component that is from about 0.1 wt. % to about 10 wt. %, from about 0.1 wt. % to about 7.5 wt. %, from about 0.1 wt. % to about 5 wt. %, from about 0.1 wt. % to about 4 wt. %, from about 0.1 wt. % to about 3 wt. %, from about 0.5 wt. % to about 10 wt. %, from about 0.5 wt. % to about 7.5 wt. %, from about 0.5 wt. % to about 5 wt. %, from about 0.5 wt. % to about 4 wt. %, from about 0.5 wt. % to about 4 wt. %, from about 1 wt. % to about 10 wt. %, from about 1 wt. % to about 7.5 wt. %, from about 1 wt. % to about 5 wt. %, from about 1 wt. % to about 4 wt. %, or from about 1 wt. % to about 3 wt. %.

Embodiment 32. The process of any one of Embodiments 1 to 31, wherein the carrier liquid component and the amphiphilic component are ingredients that are generally regarded as safe for human consumption.

Embodiment 33. The process of any one of Embodiments 1 to 32, wherein all components of the cannabinoid nanoparticle dispersion are ingredients that are generally regarded as safe for human consumption.

Embodiment 34. The process of any one of Embodiments 1 to 33, wherein the mixture and/or cannabinoid nanoparticle dispersion further comprises water.

Embodiment 35. The process of Embodiment 34, wherein the nanoparticles are at least partially dispersed in the continuous phase comprising carrier liquid component and water.

Embodiment 36. The process of Embodiment 34 or 35, wherein the cannabinoid nanoparticle dispersion is an oil in water (0/W) emulsion.

Embodiment 37. The process of any one of Embodiments 34 to 36, wherein the mixture and/or cannabinoid nanoparticle dispersion has a concentration of water that is about 1 wt. % or greater, about 2 wt. % or greater, about 3 wt. % or greater, about 4 wt. % or greater, about 5 wt. % or greater, or about 10 wt. % or greater.

Embodiment 38. The process of any one of Embodiments 34 to 37, wherein the mixture and/or cannabinoid nanoparticle dispersion has a concentration of water that is from about 1 wt. % to about 20 wt. %, from about 1 wt. % to about 15 wt. %, from about 1 wt. % to about 12 wt. %, from about 1 wt. % to about 10 wt. %, from about 5 wt. % to about 20 wt. %, from about 5 wt. % to about 15 wt. %, from about 5 wt. % to about 12 wt. %, or from about 5 wt. % to about 10 wt. %.

Embodiment 39. The process of any one of Embodiments 1 to 38, wherein the mixture and/or cannabinoid nanoparticle dispersion further comprises a fat.

Embodiment 40. The process of any one of Embodiments 1 to 39, wherein the mixture and/or cannabinoid nanoparticle dispersion further comprises a sugar, sugar alcohol, and/or sugar derivative.

Embodiment 41. The process of any one of Embodiments 1 to 40, wherein the mixture and/or cannabinoid nanoparticle dispersion further comprises a sweetener.

Embodiment 42. The process of any one of Embodiments 1 to 41, wherein the mixture and/or cannabinoid nanoparticle dispersion further comprises a triglyceride.

Embodiment 43. The process of any one of Embodiments 1 to 42, wherein the mixture and/or cannabinoid nanoparticle dispersion is free or essentially free of triglycerides.

Embodiment 44. The process of any one of Embodiments 1 to 43, wherein the mixture and/or cannabinoid nanoparticle dispersion is free or essentially free of medium chain triglyceride (MCT) oil.

Embodiment 45. The process of any one of Embodiments 1 to 44, wherein the mixture and/or cannabinoid nanoparticle dispersion further comprises a carboxylic acid.

Embodiment 46. The process of any one of Embodiments 1 to 45, wherein the mixture and/or cannabinoid nanoparticle dispersion further comprises a polycarboxylic acid.

Embodiment 47. The process of any one of Embodiments 1 to 46, wherein the mixture and/or cannabinoid nanoparticle dispersion further comprises citric acid.

Embodiment 48. The process of any one of Embodiments 1 to 47, wherein the mixture and/or cannabinoid nanoparticle dispersion further comprises a preservative.

Embodiment 49. The process of any one of Embodiments 1 to 48, wherein the cannabinoid nanoparticle dispersion is optically transparent.

Embodiment 50. The process of any one of Embodiments 1 to 49, wherein the cannabinoid nanoparticle dispersion exhibits an approximately neutral charge.

Embodiment 51. The process of any one of Embodiments 1 to 50, wherein the mixture further comprises a magnetic material.

Embodiment 52. The process of Embodiment 51, wherein the magnetic material comprises ferrite.

Embodiment 53. The process of Embodiment 51 or 52, wherein the magnetic material is coated.

Embodiment 54. The process of any one of Embodiments 1 to 53, wherein the mixture is held within a vessel at least partially constructed of a heat insulating material.

Embodiment 55. The process of any one of Embodiments 1 to 54, wherein the heat insulating material comprises glass or a ceramic.

Embodiment 56. The process of any one of Embodiments 1 to 55, wherein the vessel is cylindrical in shape.

Embodiment 57. The process of any one of Embodiments 1 to 56, further comprising rotating the vessel during exposure to the electromagnetic radiation.

Embodiment 58. The process of any one of Embodiments 1 to 57, further comprising stirring the cannabinoid nanoparticle dispersion.

Embodiment 59. A cannabinoid nanoparticle dispersion comprising:

a continuous phase comprising a carrier liquid component, and

nanoparticles at least partially dispersed in the carrier liquid component,

wherein the nanoparticles comprise a core material and a coating at least partially encapsulating the core material,

wherein the core material comprises a cannabinoid component comprising a cannabinoid and/or cannabinoid analog and the coating comprises an amphiphilic component, and

wherein the nanoparticles are characterized as having a mean particle size of about 60 nm or less, about 50 nm or less, about 40 nm or less, about 30 nm or less, about 25 nm or less, about 20 nm or less, or about 15 nm or less.

Embodiment 60. The dispersion of Embodiment 59, wherein the nanoparticles are characterized as having a mean particle size of from about 2 nm to about 60 nm, from about 5 nm to about 60 nm, from about 10 nm to about 60 nm, from about 15 nm to about 60 nm, from about 20 nm to about 60 nm, from about 2 nm to about 50 nm, from about 5 nm to about 50 nm, from about 10 nm to about 50 nm, from about 15 nm to about 50 nm, from about 20 nm to about 50 nm, from about 2 nm to about 40 nm, from about 5 nm to about 40 nm, from about 10 nm to about 40 nm, from about 15 nm to about 40 nm, from about 20 nm to about 40 nm, from about 2 nm to about 30 nm, from about 5 nm to about 30 nm, from about 10 nm to about 30 nm, from about 15 nm to about 30 nm, or from about 20 nm to about 30 nm.

Embodiment 61. The dispersion of Embodiment 59 or 60, wherein the cannabinoid component comprises at least one compound selected from the group consisting of delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), and combinations thereof.

Embodiment 62. The dispersion of any one of Embodiments 59 to 61, wherein the cannabinoid component comprises cannabidiol (CBD).

Embodiment 63. The dispersion of any one of Embodiments 59 to 62, wherein the cannabinoid component comprises tetrahydrocannabinol (THC).

Embodiment 64. The dispersion of any one of Embodiments 58 to 63, wherein the cannabinoid nanoparticle dispersion has a concentration of the cannabinoid component that is about 0.1 wt. % or greater, about 0.25 wt. % or greater, about 0.5 wt. % or greater, about 0.75 wt. % or greater, about 1 wt. % or greater, about 1.5 wt. % or greater, or about 2 wt. % or greater.

Embodiment 65. The dispersion of any one of Embodiments 59 to 64, wherein the cannabinoid nanoparticle dispersion has a concentration of the cannabinoid component that is from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1 wt. %, from about 0.5 wt. % to about 5 wt. %, from about 0.5 wt. % to about 4 wt. %, from about 0.5 wt. % to about 3 wt. %, from about 0.5 wt. % to about 2 wt. %, from about 0.5 wt. % to about 1 wt. %, from about 1 wt. % to about 5 wt. %, from about 1 wt. % to about 4 wt. %, from about 1 wt. % to about 3 wt. %, or from about 1 wt. % to about 2 wt. %.

Embodiment 66. The dispersion of any one of Embodiments 59 to 65, wherein the carrier liquid component comprises a polyol.

Embodiment 67. The dispersion of any one of Embodiments 59 to 66, wherein the carrier liquid component comprises a glycol.

Embodiment 68. The dispersion of any one of Embodiments 59 to 67, wherein the carrier liquid component comprises an alkylene glycol.

Embodiment 69. The dispersion of any one of Embodiments 59 to 68, wherein the carrier liquid component comprises a propylene glycol.

Embodiment 70. The dispersion of any one of Embodiments 59 to 69, wherein the carrier liquid component comprises a vegetable oil.

Embodiment 71. The dispersion of any one of Embodiments 59 to 70, wherein the cannabinoid nanoparticle dispersion has a concentration of the carrier liquid component that is about 40 wt. % or greater, about 50 wt. % or greater, about 60 wt. % or greater, about 70 wt. % or greater, about 80 wt. % or greater, or about 90 wt. % or greater.

Embodiment 72. The dispersion of any one of Embodiments 59 to 71, wherein the cannabinoid nanoparticle dispersion has a concentration of the carrier liquid component that is from about 40 wt. % to about 95 wt. %, from about 40 wt. % to about 90 wt. %, from about 40 wt. % to about 80 wt. %, from about 40 wt. % to about 70 wt. %, from about 40 wt. % to about 60 wt. %, from about 40 wt. % to about 50 wt. %, from about 50 wt. % to about 95 wt. %, from about 50 wt. % to about 90 wt. %, from about 50 wt. % to about 80 wt. %, from about 50 wt. % to about 70 wt. %, from about 50 wt. % to about 60 wt. %, from about 60 wt. % to about 95 wt. %, from about 60 wt. % to about 90 wt. %, from about 60 wt. % to about 80 wt. %, from about 60 wt. % to about 70 wt. %, from about 70 wt. % to about 95 wt. %, from about 70 wt. % to about 90 wt. %, from about 70 wt. % to about 80 wt. %, from about 80 wt. % to about 95 wt. %, or from about 80 wt. % to about 90 wt. %.

Embodiment 73. The dispersion of any one of Embodiments 59 to 72, wherein the amphiphilic component comprises an amphiphilic carbohydrate.

Embodiment 74. The dispersion of any one of Embodiments 59 to 73, wherein the amphiphilic component comprises an amphiphilic saccharide.

Embodiment 75. The dispersion of any one of Embodiments 59 to 74, wherein the amphiphilic component comprises an amphiphilic oligosaccharide and/or amphiphilic polysaccharide.

Embodiment 76. The dispersion of any one of Embodiments 59 to 75, wherein the amphiphilic component comprises a cyclic oligosaccharide.

Embodiment 77. The dispersion of any one of Embodiments 59 to 76, wherein the amphiphilic component comprises cyclodextrin.

Embodiment 78. The dispersion of any one of Embodiments 59 to 77, wherein the cannabinoid nanoparticle dispersion has a concentration of the amphiphilic component that is about 0.1 wt. % or greater, about 0.25 wt. % or greater, about 0.5 wt. % or greater, about 0.75 wt. % or greater, about 1 wt. % or greater, about 2 wt. % or greater, about 3 wt. % or greater, about 4 wt. % or greater, about 5 wt. % or greater, or about 10 wt. % or greater.

Embodiment 79. The dispersion of any one of Embodiments 59 to 78, wherein the cannabinoid nanoparticle dispersion has a concentration of the amphiphilic component that is from about 0.1 wt. % to about 10 wt. %, from about 0.1 wt. % to about 7.5 wt. %, from about 0.1 wt. % to about 5 wt. %, from about 0.1 wt. % to about 4 wt. %, from about 0.1 wt. % to about 3 wt. %, from about 0.5 wt. % to about 10 wt. %, from about 0.5 wt. % to about 7.5 wt. %, from about 0.5 wt. % to about 5 wt. %, from about 0.5 wt. % to about 4 wt. %, from about 0.5 wt. % to about 4 wt. %, from about 1 wt. % to about 10 wt. %, from about 1 wt. % to about 7.5 wt. %, from about 1 wt. % to about 5 wt. %, from about 1 wt. % to about 4 wt. %, or from about 1 wt. % to about 3 wt. %.

Embodiment 80. The dispersion of any one of Embodiments 59 to 79, wherein the carrier liquid component and the amphiphilic component are ingredients that are generally regarded as safe for human consumption.

Embodiment 81. The dispersion of any one of Embodiments 59 to 80, wherein all components of the cannabinoid nanoparticle dispersion are ingredients that are generally regarded as safe for human consumption.

Embodiment 82. The dispersion of any one of Embodiments 59 to 81, wherein the cannabinoid nanoparticle dispersion further comprises water.

Embodiment 83. The dispersion of Embodiment 82, wherein the nanoparticles are at least partially dispersed in the continuous phase comprising carrier liquid component and water.

Embodiment 84. The dispersion of Embodiment 82 or 83, wherein the cannabinoid nanoparticle dispersion is an oil in water (01W) emulsion.

Embodiment 85. The dispersion of any one of Embodiments 82 to 84, wherein the cannabinoid nanoparticle dispersion has a concentration of water that is about 1 wt. % or greater, about 2 wt. % or greater, about 3 wt. % or greater, about 4 wt. % or greater, about 5 wt. % or greater, or about 10 wt. % or greater.

Embodiment 86. The dispersion of any one of Embodiments 82 to 85, wherein the cannabinoid nanoparticle dispersion has a concentration of water that is from about 1 wt. % to about 20 wt. %, from about 1 wt. % to about 15 wt. %, from about 1 wt. % to about 12 wt. %, from about 1 wt. % to about 10 wt. %, from about 5 wt. % to about 20 wt. %, from about 5 wt. % to about 15 wt. %, from about 5 wt. % to about 12 wt. %, or from about 5 wt. % to about 10 wt. %.

Embodiment 87. The dispersion of any one of Embodiments 59 to 86, wherein the cannabinoid nanoparticle dispersion further comprises a fat.

Embodiment 88. The dispersion of any one of Embodiments 59 to 87, wherein the cannabinoid nanoparticle dispersion further comprises a sugar, sugar alcohol, and/or sugar derivative.

Embodiment 89. The dispersion of any one of Embodiments 59 to 88, wherein the cannabinoid nanoparticle dispersion further comprises a sweetener.

Embodiment 90. The dispersion of any one of Embodiments 59 to 89, wherein the cannabinoid nanoparticle dispersion further comprises a triglyceride.

Embodiment 91. The dispersion of any one of Embodiments 59 to 90, wherein the cannabinoid nanoparticle dispersion is free or essentially free of triglycerides.

Embodiment 92. The dispersion of any one of Embodiments 59 to 91, wherein the cannabinoid nanoparticle dispersion is free or essentially free of medium chain triglyceride (MCT) oil.

Embodiment 93. The dispersion of any one of Embodiments 59 to 92, wherein the cannabinoid nanoparticle dispersion further comprises a carboxylic acid.

Embodiment 94. The dispersion of any one of Embodiments 59 to 93, wherein the cannabinoid nanoparticle dispersion further comprises a polycarboxylic acid.

Embodiment 95. The dispersion of any one of Embodiments 59 to 94, wherein the cannabinoid nanoparticle dispersion further comprises citric acid.

Embodiment 96. The dispersion of any one of Embodiments 59 to 95, wherein the cannabinoid nanoparticle dispersion further comprises a preservative.

Embodiment 97. The dispersion of any one of Embodiments 59 to 96, wherein the cannabinoid nanoparticle dispersion is optically transparent.

Embodiment 98. The dispersion of any one of Embodiments 59 to 97, wherein the cannabinoid nanoparticle dispersion exhibits an approximately neutral charge.

Embodiment 99. A drink composition comprising a dilution of the cannabinoid nanoparticle dispersion of any one of Embodiments 59 to 98.

Embodiment 100. A food item, pharmaceutical, topical composition, or nutraceutical comprising the cannabinoid nanoparticle dispersion of any one of Embodiments 59 to 98, or dilution oEmbodiment 101. A process for preparing a drink composition comprising: preparing the cannabinoid nanoparticle dispersion according to any one of Embodiments 1 to 58; and

diluting the cannabinoid nanoparticle dispersion with a liquid comprising water.

Embodiment 102. A process for preparing a food item, pharmaceutical, topical composition, or nutraceutical comprising:

preparing the cannabinoid nanoparticle dispersion according to any one of Embodiments 1 to 58; and

mixing the cannabinoid nanoparticle dispersion or dilution or concentrate thereof with one or more ingredients of the food item, pharmaceutical, topical composition, or nutraceutical.

Embodiment 103.A method of treating a medical condition in a subject in thereof comprising administering to the subject a pharmaceutical composition comprising the cannabinoid nanoparticle dispersion of any one of Embodiments 59 to 98, or dilution or concentrate thereof.

Embodiment 104. The method of Embodiment 103, wherein the medical condition is selected from the group consisting of neuropathic pain, pain related to cancer and trauma, spasticity associated with multiple sclerosis, fibromyalgia, gastrointestinal, metabolic, neurological, circulatory, soft tissue, musculoskeletal, chronic or acute pain, nausea, decreased appetite, skin disorders, sexual dysfunction, glaucoma, AIDS wasting, neuropathic pain, treatment of spasticity associated with multiple sclerosis, fibromyalgia, chemotherapy-induced nausea, allergies, inflammation, infection, epilepsy, depression, migraine, bipolar disorders, anxiety disorder, dependency and withdrawal, and combinations thereof.

Embodiment 105. The method of Embodiment 103, wherein the medical condition is to alleviate, or relief symptoms or side effects associated with anti-retroviral therapy, chemotherapy and radiation therapy.

Embodiment 106. The method of Embodiment 103, wherein the medical condition a viral infection or a symptom such as lung inflammation caused by viral infections.

Embodiment 107. The method of Embodiment 103, wherein the viral infection is caused by a coronavirus, SARS-CoV-2, SARS-CoV, MERS-CoV, and/or influenza.

### EXAMPLES

The following non-limiting examples are provided to further illustrate the present disclosure.

### Example 1

An oil phase solution, including cannabinoid or cannabinoid analog, and a water phase solution are combined to form a cannabinoid or cannabinoid analog formulation solution. The percentages described below are by mass and are with respect to the cannabinoid or cannabinoid analog formulation solution.

Oil Phase Preparation

Propylene glycol (38%) is added to a container and stirred using a mechanical or magnetic stirrer. MCT oil (0.25%) is added to the propylene glycol, then deionized water (3%) is added and stirred until a consistent solution. Cannabinoid powder from isolate (2.6%) is then added and mixture is stirred at 3,000 rpm for 30 seconds. Reb-M from Stevia leaf (3%) is added after the cannabinoid powder and the mixture is stirred for an additional 15 seconds. Next, inner aloe leaf (3%) is added and the mixture is stirred for an additional 10 seconds.

The above cannabinoid mixture is placed in a conventional microwave and microwaved for 75 seconds on a rotating plate. The cannabinoid mixture/solution is removed from the microwave and immediately stirred for 30 seconds to fully dissolve the cannabinoid nanoparticles (nanoparticles less than 25 nm in size) in the solvent. The cannabinoid solvent is then refrigerated for 2 hours to allow fully encapsulation to form.

Water Phase Preparation

In a container separate from the container of the oil phase solution, propylene glycol (40%), clustered water (7%) and vegetable glycerin (10%) are added and stirred with a magnetic stirred at 3,000 rpm for 60 seconds until the solution becomes clear and consistent.

Cannabinoid Solution Preparation

The oil phase solution is stirred with a magnetic stirrer at 1,000 rpm. The water phase solution is poured directly down the vortex of the oil phase solution in the oil phase solution container while slowly increasing the rpm of the magnetic stirred to 3,000 rpm. After pouring the entire water phase solution into the oil phase solution, including a magnetic stirred in the container of the water phase solution, the combined solution is stirred for an additional 5 minutes at 3,000 rpm using the two magnetic stirrers. The two magnetic stirrers create electromagnetic charges that form smaller molecules similar to how the clustered water is formed.

### Example 2

A cannabinoid nanoparticle dispersion was prepared according to the following procedure. Cannabidiol, propylene glycol, water, cyclodextrin, citric acid, and a blend of sugars/sweeteners were combined in a glass beaker and mixed for about 30 seconds using a magnetic stir bar. After stirring, the beaker containing the mixture and stir bar was placed in an inverter microwave oven and heated for 45 seconds to a temperature of approximately 88° C. to form the dispersion. Following heating, the mixture was mixed for 1 minute using the magnetic stir bar. After settling, the mixture was filtered. The composition of the dispersion is presented in Table 1.

TABLE 1

| Ingredient | Concentration (wt. %) |
|---|---|
| cannabidiol, (−)-trans- | 1.25 |
| cannabidiol, (CBD) | |
| Propylene Glycol | 83 |
| Deionized Water | 7 |
| Cyclodextrin | 3 |
| Citric Acid | 2 |
| Other Sugars, Sugar | 3.75 |
| Derivatives, and Sweeteners | |
| (Total) | |

The cannabinoid nanoparticle dispersion was analyzed for various physical parameters as presented in Table 2. Analysis showed results very similar to pure water. The zeta potential of water is 0±4 mV. The refractive index of water is 1.33. The particle size of water is 0.2 nm. The conductivity of water is 0.5 for purified and >5 for tap.

The analytical instruments and methodology are provided below.

TABLE 2

| Parameter | Result | | Methods | Instrument |
|---|---|---|---|---|
| Mean Particle Size | 11 | nanometers | Dynamic Light | Malvern Zetasizer Nano |
| (1) | | | Scattering | ZS |
| Mean Particle Size | 14 | nanometers | Static Multiple Light | Turbiscan Lab & ASG |
| (2) | | | Scattering | |
| Particle Shape | Sphere | | Dynamic Light | Malvern Zetasizer Nano |
| | | | Scattering | ZS |
| Refractive Index | 1.43 | | Turbidity | Refractometer |
| Z-Average | 53.9 | | Dynamic Light | Malvern Zetasizer Nano |
| | | | Scattering | ZS |
| Zeta Potential | −3.59 | mV | Clear Disposable | Malvern Zetasizer Nano |
| | | | Zeta Cell | ZS |
| Volume Fraction | 1.44% | | Multiple Light | Turbiscan Lab & ASG |
| | | | Scattering | |
| | | | Technology | |
| Conductivity | 0.12 | mS/cm | Dynamic Light | Malvern Zetasizer Nano |
| | | | Scattering | Zs |
| PDI | 0.41 | | Dynamic Light | Malvern Zetasizer Nano |
| | | | Scattering | ZS |
| Solubility | Soluble in Water | | Turbidity | Turbiscan Lab & ASG |
| Stability | 12 | Months | Quantitative Analysis | Qualitative Analysis |

Malvern Zetasizer Nano ZS: High performance two angle particle and molecular size analyzer for the enhanced detection of aggregates and measurement of small or dilute samples, and samples at very low or high concentration using dynamic light scattering with ‘NIBS’ optics. incorporates three techniques in a single compact unit and has a range of options and accessories to optimize and simplify the measurement of different sample types.

Dynamic Light Scattering: Used to measure particle and molecule size. This technique measures the diffusion of particles moving under Brownian motion and converts this to size and a size distribution using the Stokes-Einstein relationship. Non-Invasive Back Scatter technology (NIBS) is incorporated to give the highest sensitivity simultaneously with the highest size and concentration range. Measurement of size as a function of concentration enables the calculation of k_{D}, the DLS interaction parameter.

Laser Doppler Micro-Electrophoresis: Used to measure zeta potential. An electric field is applied to a solution of molecules or a dispersion of particles, which then move with a velocity related to their zeta potential. This velocity is measured using a laser interferometric technique called M3-PALS (Phase analysis Light Scattering). This enables the calculation of electrophoretic mobility, and from this the zeta potential and zeta potential distribution. A surface zeta potential accessory uses tracer particles to measure electro-osmosis close to a sample surface to calculate the zeta potential of the surface.

Turbiscan Lab & ASG: Enables fast and sensitive identification of destabilization mechanisms such as creaming, sedimentation, flocculation and coalescence. A temperature-controlled measurement cell allows either stability monitoring at specific storage temperatures or accelerating destabilization process. This robust system designed to measure the average particle size in concentrated solutions, using S-MLS technology to quantify the long-term stability of formulations without dilution or mechanical stress.

### Example 3

Aqueous beverages were prepared by mixing cannabinoid nanoparticle dispersion with OXIGEN water (a high alkaline drinking water product, pH 7.2-7.4) prepared in accordance with Example 2. Beverages at three different cannabinoid concentrations were prepared to show solubility of the dispersion and optical clarity at increased potency levels.

The beverages were analyzed for various physical parameters as presented in Table 3.

TABLE 3

| Parameter | Values | Methods | Instrument |
|---|---|---|---|
| Appearance | Clear Liquid | Organoleptic Test | Sensory |
| Color | Transparent, | Organoleptic Test | Sensory |
| | Colorless | | |
| Odor | Odorless | Organoleptic Test | Sensory |
| Taste | No Off Tastes | Organoleptic Test | Sensory |
| Total THC | None Detected | SOP-TECH-001 | UPLC-DAD |
| Total CBD | 0.0062 mg/ml | SOP-TECH-001 | UPLA-DAD |
| (3 mgs) | (0.0006 wt. %) | | |
| *Total CBD | 0.0178 mg/ml | SOP-TECH-001 | UPLC-DAD |
| (8 mgs) | (0.0018 wt. %) | | |
| *Total CBD | 0.1035 ml/ml | SOP-TECH-001 | UPLS-DAD |
| (51 mgs) | (0.0091 wt. %) | | |
| Particle Size | <35 nanometers | Multiple Light | Turbiscan Lab & ASG |
| | | Scattering Technology | Autosampler |
| Volume | 0.003% | Multiple Light | Turbiscan Lab & ASG |
| Fraction | | Scattering Technology | Autosampler |

UPLC-DAD-MS: An ultra-high-performance liquid chromatography-diode array detector-tandem mass spectrometry (UPLC-DAD-MS) method was established for the characterization of the active ingredient.

### Example 4

The cannabinoid nanoparticle dispersion (CBD concentrate) prepared in accordance with Example 2 and the aqueous beverage (CBD OXIGEN water) prepared in accordance with Example 3 were subjected to various stability tests.

Samples were analyzed using the Turbiscan Lab stability analyzer and accompanying AGS autosampler. This instrument utilizes Static Multiple Light Scattering to detect changes in particle size and concentration within emulsions and suspensions to determine the shelf stability and overall quality of the formulation. A NIR light source (880 nm) is appended to a mobile reading head and is pulsed into a sample within a glass measuring cell every 20 μm vertically. The amount of transmitted (T) and backscattered (BS) light are acquired by detectors at 180° and 45° to the light source as the amount of scattered light is directly dependent upon particle size and concentration in accordance with Mie scattering theory. By making these measurements over time, changes in the backscatter and transmission profiles correspond particle migration in the sample (settling and creaming) as well as particle size change (flocculation). This technology accelerates the aging process 200 times faster than the naked eye and is similar to QC used in pharmaceuticals.

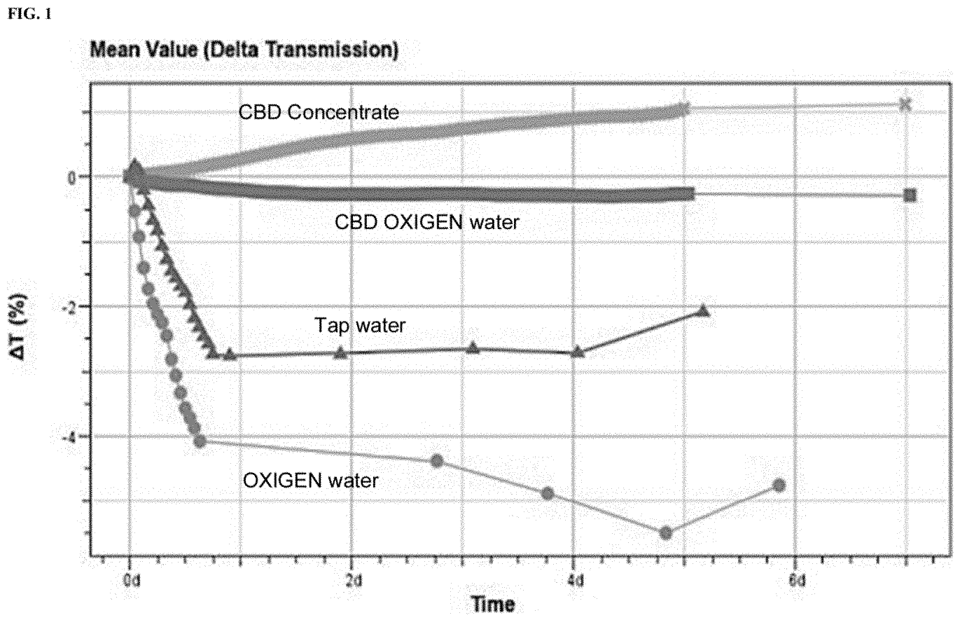

Approximately 20 mL of each sample was transferred into 30 mL glass vials. Samples were then scanned every hour for 18-24 h at room temperature to analyze the stability of the compositions listed in Table 4. FIG. 1 shows the change in transmission signal of the CBD concentrate and dosed beverage (CBD OXIGEN water) at room temperature compared to local tap water and the un-dosed beverage (OXIGEN water).

TABLE 4

| Sample | Temperature | Scan Frequency |
|---|---|---|
| Blank Concentrate | RT; 40° C. | 1 scan each hour for the first 24 |
| Tap Water | RT | hours; then 1 scan per day for 35 |
| OXIGEN Water | RT | days. |
| CBD Concentrate | RT; 40° C. | |
| CBD OXIGEN | RT; 40° C. | |
| Water | | |

Turbiscan technology enables a fast detection of all the destabilization phenomena in just few days of analysis. Comparison and ranking samples can be done by the detection and quantification of destabilization phenomena and TSI algorithm that considers various destabilization mechanisms to quantify the kinetics and assess sedimentation, particle size and global destabilization with the TSI.

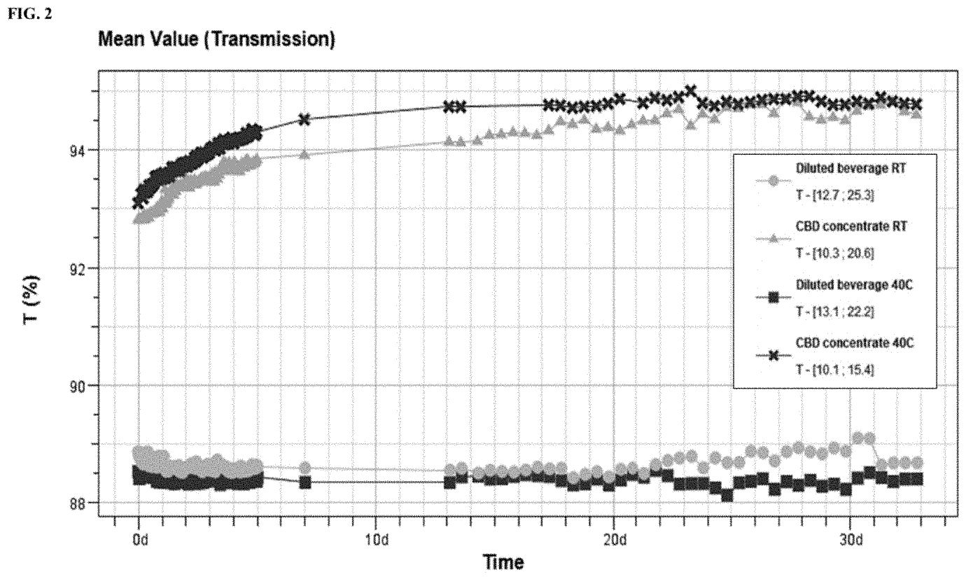

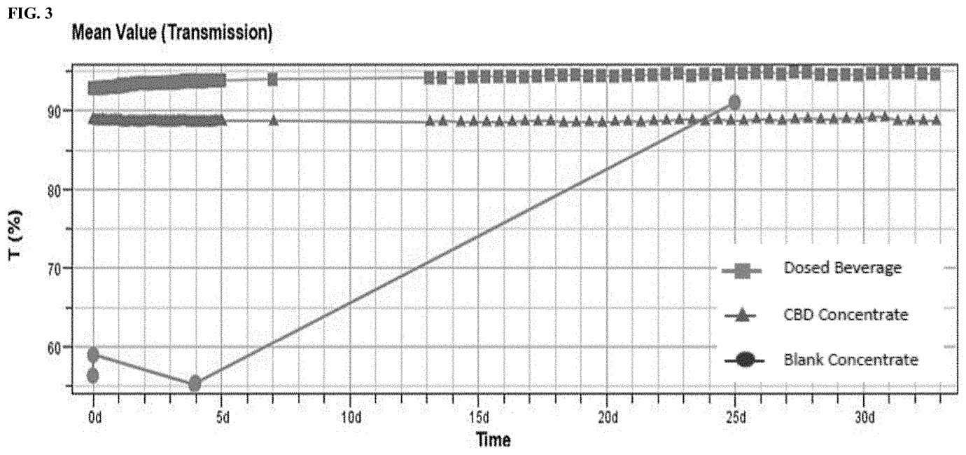

The raw transmission signal of the CBD concentrate and CBD OXIGEN water at room temperature and 40° C. over the first 35 days of analysis are presented in FIG. 2 and FIG. 3. Comparatively, similar products see a slope after only one day. Particle size was also evaluated after 35 days of storage at room temperature and 40° C. The results are presented in Table 5.

TABLE 5

| | Time and | Volume | Particle size | |
|---|---|---|---|---|
| SAMPLE | Temperature | fraction, Φ | (range) | |
| CBD Concentrate | Day 1 - RT | 1.44 | <15 | nm |
| | Day 35 - RT | 1.44 | 15-21 | nm |
| | Day 1 - 40° C. | 1.44 | <15 | nm |
| | Day 35 - 40° C. | 1.44 | 15-21 | nm |
| CBD OXIGEN Water | Day 1 - RT | 0.003 | <36 | nm |
| | Day 35 - RT | 0.003 | 36-46 | nm |
| | Day 1 - 40° C. | 0.003 | <36 | nm |
| | Day 35 - 40° C. | 0.003 | 36-46 | nm |

The results show that no new destabilization mechanism was observed upon dilution of the CBD concentrate with the OXIGEN bottle water product. Almost no changes were observed in transmission of the diluted CBD and the CBD concentrate over the long-term study of 35 days. The control samples of tap water and OXIGEN water showed more changes in their transmission profiles than the CBD concentrate and CBD-dosed water sample over the analysis period. The particle sizes of the concentrate and the CBD OXIGEN Water showed similar particle sizes over time. The physical stability data show that dosing of the CBD concentrate into OXIGEN water does not significantly alter the shelf-life of the existing product assuming similar trends in chemical stability.

Test were also conducted to evaluate real time stability. All samples were analyzed at Cannalysis Labs using a UPLC-DAD-MS for the characterization of the active ingredient. Batch samples were placed in glass vials and stored at room tempurature. The goal was to monitor change in potency over time to assure accurate labeling of finsihed products, show consistant potency levels from multiple batches, and have a reliable chemical profile free from THC, pathogens, heavy metals, microbrials, mycotoxins and chemical residue.

Samples of the CBD concentrate were evaluated for over various storage periods. The results are presented in Table 6.

TABLE 6

| Test Period | CBD potency | Sample Container |
|---|---|---|
| 2 months after manufacture | 14.10 mg/ml | Clear Glass Vial |
| 7 months after manufacture | 12.54 mg/ml | Opaque Glass Vial |
| 10 months after manufacture | 11.91 mg/ml | Clear Glass Vial |

No significant impact when observed using opaque container vs. clear container. No change in appearance, odor or taste was observed.

Samples of CBD concentrates manufactured on different dates and at three different concentrations were also analyzed for chemical consistency. The results are presented in Table 7 (same CBD concentrations, different manufacturing dates) and Table 8 (different CBD concentrations and different manufacturing dates).

TABLE 7

| Parameter | Sample 1 | Sample 2 | Instrument | Methods |
|---|---|---|---|---|
| THC | ND | ND | UPLC-DAD | SOP-TECH-001 |
| Heavy Metals | PASS | PASS | ICP-MS | SOP-TECH-013 |
| Chemical | PASS | PASS | LC-MS/MS | SOP-TECH-002 |
| Residue | | | | |
| Chemical | PASS | PASS | GC-MS/MS | SOP-TECH-010 |
| Residue GC | | | | |
| Microbial | PASS | PASS | qPCR | SOP-TECH-016, |
| | | | | 022 |
| Mycotoxins | PASS | PASS | LC-MS/MS | SOP-TECH-020 |

TABLE 8

| Parameter | Sample 3 | Sample 4 | Sample 5 | Instrument | Methods |
|---|---|---|---|---|---|
| CBD Content | 21.87 | 28 | 15.91 | UPLC-DAD | SOP-TECH-001 |
| | mg/ml | mg/ml | mg/ml | | |
| THC | ND | ND | ND | UPLC-DAD | SOP-TECH-001 |
| Heavy Metals | PASS | PASS | PASS | ICP-MS | SOP-TECH-013 |
| Chemical | PASS | PASS | PASS | LC-MS/MS | SOP-TECH-002 |
| Residue | | | | | |
| Chemical | PASS | PASS | PASS | GC-MS/MS | SOP-TECH-010 |
| Residue GC | | | | | |
| Microbial | PASS | PASS | PASS | qPCR | SOP-TECH-016, |
| | | | | | 022 |
| Mycotoxins | PASS | PASS | PASS | LC-MS/MS | SOP-TECH-020 |

Samples of CBD concentrates manufactured on different dates and at two different concentrations were also analyzed for batch to batch reproducibility. The results are presented in Table 9.

TABLE 9

| Batch ID | CBD Potency |
|---|---|
| 190927A | 21.04 mg/ml |
| 191118A | 21.87 mg/ml |
| 190820A | 28.35 mg/ml |
| 191017A | 28.75 mg/ml |

Modifications and variations of the disclosed embodiments are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above compositions, processes, and methods without departing from the scope of the invention, it is intended that all matter contained in the above description and shown in the accompanying drawings shall be interpreted as illustrative and not in a limiting sense.

What is claimed is:

1. A process for preparing a cannabinoid nanoparticle dispersion, the process comprising:
exposing a mixture comprising a carrier liquid component, an amphiphilic component, and a cannabinoid component comprising a cannabinoid and/or cannabinoid analog to electromagnetic radiation in a heating zone to form the cannabinoid nanoparticle dispersion comprising nanoparticles at least partially dispersed in a continuous phase comprising at least a portion of the carrier liquid component,
wherein the nanoparticles comprise a core material and a coating at least partially encapsulating the core material,
wherein the core material comprises at least a portion of the cannabinoid component and the coating comprises at least a portion of the amphiphilic component, and
wherein the nanoparticles are characterized as having a mean particle size of about 60 nm or less, about 50 nm or less, about 40 nm or less, about 30 nm or less, about 25 nm or less, about 20 nm or less, or about 15 nm or less.

2. The process of claim 1, wherein the nanoparticles are characterized as having a mean particle size of from about 2 nm to about 60 nm, from about 5 nm to about 60 nm, from about 10 nm to about 60 nm, from about 15 nm to about 60 nm, from about 20 nm to about 60 nm, from about 2 nm to about 50 nm, from about 5 nm to about 50 nm, from about 10 nm to about 50 nm, from about 15 nm to about 50 nm, from about 20 nm to about 50 nm, from about 2 nm to about 40 nm, from about 5 nm to about 40 nm, from about 10 nm to about 40 nm, from about 15 nm to about 40 nm, from about 20 nm to about 40 nm, from about 2 nm to about 30 nm, from about 5 nm to about 30 nm, from about 10 nm to about 30 nm, from about 15 nm to about 30 nm, or from about 20 nm to about 30 nm.

3. The process of claim 1, further comprising generating the electromagnetic radiation.

4. The process of claim 1, wherein the electromagnetic radiation comprises microwave radiation.

5. The process of claim 4, wherein the microwave radiation is generated by electrical power and the electrical power is linearly controlled.

6. The process of claim 5, wherein the electrical power is not pulse controlled.

7. The process of claim 4, wherein the microwave radiation is generated by an inverter microwave oven.

8. The process of claim 4, wherein the microwave radiation has a frequency that is 500 MHz to about 5 GHz, or from about 700 MHz to about 4 GHz, from about 900 MHz to about 3 GHz, from about 1 GHz to about 3 GHz, or from about 1.5 GHz to about 2.5 GHz.

9. The process of claim 4, wherein during exposure, the microwave radiation is continuous.

10. The process of claim 4, wherein during exposure, the microwave radiation is not intermittent or pulsed.

11. The process of claim 4, wherein during exposure, the frequency of the microwave radiation is 300 MHz or greater, 500 MHz or greater, or 1 GHz or greater.

12. The process of claim 1, wherein the mixture is heated to a temperature of from about 75° C. to about 100° C., from about 75° C. to about 95° C., or from about 80° C. to about 95° C. by exposure to the electromagnetic radiation.

13. The process of claim 1, wherein the cannabinoid component comprises at least one compound selected from the group consisting of delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), and combinations thereof.

14. The process of claim 1, wherein the cannabinoid component comprises cannabidiol (CBD).

15. A cannabinoid nanoparticle dispersion comprising:
a continuous phase comprising a carrier liquid component, and
nanoparticles at least partially dispersed in the carrier liquid component,
wherein the nanoparticles comprise a core material and a coating at least partially encapsulating the core material,
wherein the core material comprises a cannabinoid component comprising a cannabinoid and/or cannabinoid analog and the coating comprises an amphiphilic component, and
wherein the nanoparticles are characterized as having a mean particle size of about 60 nm or less, about 50 nm or less, about 40 nm or less, about 30 nm or less, about 25 nm or less, about 20 nm or less, or about 15 nm or less.

16. A drink composition comprising a dilution of the cannabinoid nanoparticle dispersion of claim 15.

17. A food item, pharmaceutical, topical composition, or nutraceutical comprising the cannabinoid nanoparticle dispersion of claim 15 or dilution or concentrate thereof.

18. A process for preparing a drink composition comprising:
preparing the cannabinoid nanoparticle dispersion according to claim 1; and
diluting the cannabinoid nanoparticle dispersion with a liquid comprising water.

19. A process for preparing a food item, pharmaceutical, topical composition, or nutraceutical comprising:
preparing the cannabinoid nanoparticle dispersion according to claim 1; and
mixing the cannabinoid nanoparticle dispersion or dilution or concentrate thereof with one or more ingredients of the food item, pharmaceutical, topical composition, or nutraceutical.

20. A method of treating a medical condition in a subject in thereof comprising administering to the subject a pharmaceutical composition comprising the cannabinoid nanoparticle dispersion of claim 15, or dilution or concentrate thereof.
